(19) United States
(12) Patent Application Publication
(10) Pub. No.: US 2016/0192843 A1
(43) Pub. Date: Jul. 7, 2016

(54) PHOTOACOUSTIC APPARATUS

(71) Applicant: CANON KABUSHIKI KAISHA, Tokyo (JP)

(72) Inventor: Robert A Kruger, Oriental, NC (US)

(21) Appl. No.: 14/916,162

(22) PCT Filed: Sep. 3, 2014
(86) PCT No.: PCT/US14/53820

§ 371 Date: Mar. 2, 2016

Related U.S. Application Data
(60) Provisional application No. 61/873,542, filed on Sep. 4, 2013.

(51) Int. Cl. A61B 5/00

(52) U.S. Cl. CPC A61B 5/0095

(57) ABSTRACT

A photoacoustic apparatus disclosed in the description includes a light source; transducers that detect acoustic waves and output electric signals, the acoustic waves being generated when an object is irradiated with light generated from the light source; a support member that supports the transducers such that directional axes of the transducers gather; a moving unit that moves the support member relative to the object within a movement region; a storage unit that stores the electric signals output from the transducers at timings; and a computing unit that acquires object information for each reconstruction position on the basis of the electric signals stored in the storage unit. The light source generates the light at the timings. The moving unit moves the support member such that there exists a region in which a density of a distribution of positions of the support member at the timings is constant.

### TECHNICAL FIELD

The present invention relates to a photoacoustic apparatus that acquires information regarding the interior of an object by making use of a photoacoustic effect.

### BACKGROUND ART

Studies of optical imaging apparatuses have been actively conducted in the field of medicine. The optical imaging apparatuses irradiate an object (such as a living body) with light from a light source (such as a laser) and form an image from information regarding the interior of the object, the information being acquired on the basis of incident light. Photoacoustic imaging (PAI) is one of such optical imaging techniques. In the photoacoustic imaging, an object is irradiated with pulsed light generated from a light source, acoustic waves (typically ultrasonic waves) generated from tissues of the object that absorb energy of the pulsed light that has propagated and that has been diffused in the object are detected, and information regarding the interior of the object is subjected to imaging on the basis of the detection signals. That is, by making use of a difference in the rate of absorption of optical energy between a target area (such as a tumor) and other tissues, an acoustic wave detector receives elastic waves (photoacoustic waves) generated when a test area momentarily expands by absorbing optical energy with which the test area is irradiated. By mathematically processing the detection signals, it is possible to acquire information regarding the interior of the object. In recent years, the photoacoustic imaging has been used to actively conduct preclinical studies in which blood vessels of small animals are imaged, and clinical studies in which the principle of the photoacoustic imaging is applied to the diagnosis of, for example, breast cancer (“Photoacoustic imaging in biomedicine”, M. Xu, L. V. Wang, REVIEW OF SCIENTIFIC INSTRUMENT, 77, 041101, 2006).

However, it is desired that photoacoustic apparatuses more efficiently and precisely acquire object information.

### CITATION LIST

### Non Patent Literature

NPL 1 Photoacoustic Imaging in Biomedicine

### SUMMARY OF INVENTION

The present invention provides a photoacoustic apparatus that is capable of efficiently and precisely acquiring object information.

A photoacoustic apparatus that is disclosed in the description includes a light source; a plurality of transducers configured to detect acoustic waves and output electric signals, the acoustic waves being generated when an object is irradiated with light generated from the light source; a support member configured to support the plurality of transducers such that directional axes of the plurality of transducers gather; a moving unit configured to move the support member relative to the object within a movement region; a storage unit configured to store the electric signals output from the plurality of transducers at a plurality of timings; and a computing unit configured to acquire object information for each reconstruction position on the basis of the electric signals stored in the storage unit. The light source generates the light at the plurality of timings. The moving unit moves the support member such that there exists a region in which a density of a distribution of positions of the support member at the plurality of timings is constant.

Further features of the present invention will become apparent from the following description of exemplary embodiments with reference to the attached drawings.

### BRIEF DESCRIPTION OF DRAWINGS

FIG. 1 schematically illustrates an exemplary configuration of a photoacoustic apparatus according to an embodiment.

FIG. 2 schematically illustrates an exemplary movement of a support member in the embodiment.

FIG. 3 illustrates an exemplary movement path of the support member in the embodiment.

FIG. 4 illustrates another exemplary movement path of the support member in the embodiment.

FIG. 5 illustrates a distribution of measurement positions in the embodiment.

FIG. 6 illustrates a photoacoustic apparatus according to Example.

FIG. 7 schematically illustrates part of a configuration of the photoacoustic apparatus according to Example.

FIG. 8 schematically illustrates an exemplary configuration of the photoacoustic apparatus according to Example.

FIG. 9 illustrates distributions of measurement positions in the photoacoustic apparatus according to Example.

FIG. 10 illustrates photoacoustic images obtained by the photoacoustic apparatus according to Example.

FIG. 11 illustrates a phantom used in Example.

FIG. 12 illustrates photoacoustic images obtained by the photoacoustic apparatus according to Example.

FIGS. 13A and 13B show a result of analysis of the photoacoustic images obtained by the photoacoustic apparatus according to Example.

FIG. 14 illustrates a different photoacoustic image obtained by the photoacoustic apparatus according to Example.

FIG. 15 illustrates a different photoacoustic image obtained by the photoacoustic apparatus according to Example.

### DESCRIPTION OF EMBODIMENTS

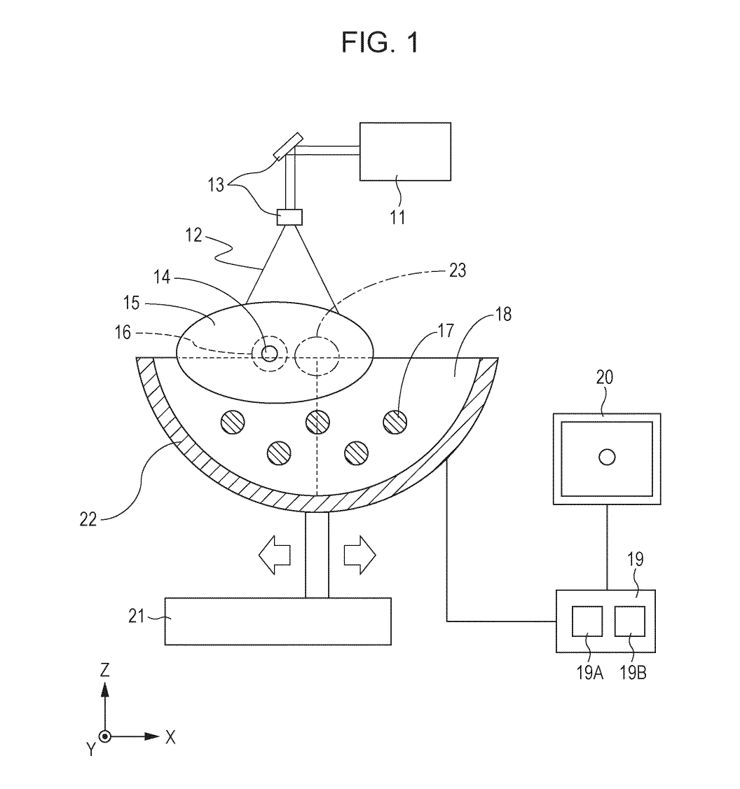

A configuration of a photoacoustic apparatus 1 according to an embodiment is described with reference to FIG. 1.

The photoacoustic apparatus 1 according to the embodiment includes a light source 11, an optical system 13, a plurality of transducers 17 that are supported by a support member 22, a computer 19, a display device 20, and a scanner 21.

Pulsed light 12 emitted from the light source 11 is guided while being processed into a desired light distribution shape by the optical system 13 including, for example, a lens, a mirror, an optical fiber, and a diffusing plate. Then, the pulsed light 12 is applied to an object 15, such as a living body. At a timing in which the pulsed light 12 is applied, the pulsed light 12 reaches the entire interior of the object 15 at substantially the same time. When energy of the pulsed light 12 that has propagated through the interior of the object 15 is partly absorbed by a light absorber 14 (which eventually becomes a sound source), such as a blood vessel containing a large amount of hemoglobin, photoacoustic waves (typically ultrasonic waves) 16 are generated by thermal expansion of the light absorber 14. The photoacoustic waves 16 propagate through the interior of the object 15 and an acoustic matching material 18, and reach the plurality of transducers 17 supported by the support member 22. The plurality of transducers 17 receive the photoacoustic waves 16 and convert them into electric signals.

While the scanner 21 moves the support member 22, the photoacoustic waves are measured at a plurality of timings. The term “measure” in the description refers to application of light and reception of photoacoustic waves generated by the application of light. The term “measurement position” refers to the position of a search unit when light is applied, that is, the position of the support member 22. In the embodiment, a center position of the support member 22 at a light irradiation timing serves as a measurement position.

Typically, the speed of propagation of the photoacoustic waves 16 is faster than the speed at which the scanner 21 moves the support member 22. Therefore, the photoacoustic waves 16 are received at positions of the plurality of transducers 17 at the timing in which the pulsed light 12 is applied. Here, the movement of the support member 22 from the time when the pulsed light 12 is applied to the object 15 to the time when the plurality of transducers 17 detect the photoacoustic waves 16 can be ignored. Therefore, in the present embodiment, the timing in which the pulsed light 12 is applied corresponds to the timing in which the photoacoustic waves 16 are measured (hereunder referred to as “measurement timing”). In addition, the positions taken by the plurality of transducers 17 at the timing in which the pulsed light 12 is applied correspond to photoacoustic-wave measurement positions that can be taken at the timing in which the photoacoustic waves 16 are measured. Since the support member 22 supports the plurality of transducers 17, the positions of the plurality of transducers 17 can be specified by specifying the position of the support member 22.

Next, electric signals output from the plurality of transducers 17 at each timing are amplified and converted into digital signals by the computer 19, and are stored in a storage unit in the computer 19. That is, the storage unit in the computer 19 stores the electric signals output from the plurality of transducers 17 at different timings.

Then, the computer 19 acquires object information for each reconstruction position in a region subjected to imaging using the electric signals output from the plurality of transducers at the different timings. A reconstruction position where the object information is acquired is a voxel in the case where three-dimensional information is acquired, and is a pixel in the case where two-dimensional information is acquired. Known reconstruction techniques, such as universal back projection (UBP) and filtered back projection (FBP), can be used to acquire object information from the electric signals. According to these reconstruction techniques, it is possible to acquire object information for one position from the electric signals obtained at the different timings.

In the present embodiment, the entire object is set as a region to be subjected to imaging. A region that has been previously set or that has been set by a user using an input unit can be set as a region to be subjected to imaging.

Next, the computer 19 generates image data for being displayed on the display device 20 from the acquired object information.

Next, the computer 19 displays the image data on the display device 20. The image of the object information displayed on the display device 20 in this way can be used for, for example, diagnostic purposes.

To acquire object information for each position in a region to be subjected to imaging, the computer 19 need not use all electric signals stored in the storage unit. That is, from among the electric signals stored in the storage unit, the computer 19 may use electric signals output from the plurality of transducers 17 at a part of the timings to acquire object information for each position in the region to be subjected to imaging. For example, electric signals used to acquire object information may be determined on the basis of the directionality of the transducers or the light value for each position at each timing.

In the present embodiment, since wave-number information can be efficiently acquired, it is possible to precisely acquire object information from a large amount of wave-number information for each position even in the case where electric signals obtained at a part of the timings are used. In addition, since the number of electric signals used to acquire object information is reduced, it is possible to reduce the time required to acquire the object information.

Examples of the object information that can be acquired by the photoacoustic apparatus according to the embodiment include a distribution of initial sound pressures of photoacoustic waves, a distribution of optical energy absorption densities, a distribution of absorption coefficients, and a distribution of concentrations of materials that form the object. The concentrations of materials include a degree of oxygen saturation, an oxyhemoglobin concentration, a deoxyhemoglobin concentration, and a total hemoglobin concentration. The total hemoglobin concentration is the sum of the concentrations of oxyhemoglobin and deoxyhemoglobin.

### Each Component of Photoacoustic Apparatus

Next, each component of the photoacoustic apparatus according to the present embodiment is described in detail.

### Light Source 11

The light source 11 supplies optical energy to the object 15 and causes the photoacoustic waves 16 to be generated. When the object 15 is a living body, the light source 11 emits light of a specific wavelength to be absorbed by a specific one of components of the object 15. It is desirable that the light source 11 be a pulsed light source that can generate pulsed light of the order of from a few to a few hundred nanoseconds as irradiation light. More specifically, it is desirable to use a pulse width of approximately 10 to 100 nanoseconds to efficiently generate photoacoustic waves. It is desirable to use laser as the light source 11 to achieve high output. However, a light-emitting diode or the like may be used instead of the laser. Various lasers, such as a solid-state laser, a gas laser, a fiber laser, a dye laser, and a semiconductor laser, may be used for the laser. For example, the irradiation timing, waveform, and intensity are controlled by a light-source controller (not shown). When the object 15 is a living body, it is desirable that the wavelength of the light source 11 used be one that allows light to propagate to the interior of the living body. Specifically, the wavelength may range from 500 nm to 1200 nm.

The light source 11 may be provided separately from the photoacoustic apparatus. In addition, the light source 11 may be formed by either a single light source or a plurality of light sources.

### Optical System 13

The pulsed light 12 emitted from the light source 11 is guided to the object 15 while being processed into a desired light distribution shape typically by the optical system 13 including, for example, a lens or a mirror. For example, an optical waveguide such as an articulating arm formed by mounting a mirror or the like in a lens barrel, optical fibers, and a bundle of optical fibers are regarded as components of the optical system 13. Examples of other components of the optical system 13 include a mirror that reflects light, a lens that converges or diverges light or changes the shape of light, and a diffusing plate that diffuses light. Any optical components may be used, as long as the pulsed light 12 emitted from the light source 11 is applied to the object 15 in a desired shape. It is desirable to diverge the pulsed light 12 by the lens over a larger area, rather than converging the pulsed light 12 by the lens, from the viewpoint of expanding a region of the object 15 to be diagnosed.

The photoacoustic apparatus need not include the optical system 13 if a pulsed light 12 that is desired pulsed light is emitted from the light source 11.

### Object 15 and Light Absorber 14

The object 15 and the light absorber 14 are now described, although they do not constitute part of the photoacoustic apparatus according to the present embodiment. The photoacoustic apparatus according to the present embodiment is primarily used, for example, for diagnosis of malignant tumors or blood vessel diseases of humans or animals, and follow-up of chemotherapy. Therefore, the object 15 may be an area of a human or animal body to be diagnosed, such as a breast, a finger, an arm, or a leg. The light absorber 14 inside the object 15 has a relatively high absorption coefficient therein. For example, when a human body is an object to be measured, the light absorber 14 may be oxygenated or reduced hemoglobin, or a blood vessel or a newborn blood vessel containing a large amount of oxygenated or reduced hemoglobin. The light absorber 14 on the surface of the object 15 may be, for example, melanin. By selecting an appropriate wavelength of light, other materials, such as fat, water, and collagen, may serve as the light absorber 14 in a human body.

### Transducer 17

A transducer 17 receives acoustic waves and converts them into electric signals which are analog signals. The transducer 17 may be any transducer as long as it detects photoacoustic waves, such as a transducer that uses a piezoelectric effect, a transducer that uses resonance of light, and a transducer that makes use of changes in capacitance. A plurality of transducers 17 are arranged in the present embodiment. By the use of such multi-dimensionally arranged elements, acoustic waves can be received simultaneously at multiple locations. This can reduce measurement time and reduce the influence of, for example, vibration of the object 15.

### Support Member 22

The support member 22 supports the plurality of transducers 17 along the support member 22. FIG. 1 is a sectional view of the support member 22 in an x-z plane thereof.

It is desirable that the support member 22 support the plurality of transducers 17 along a closed surface that surrounds the object 15. However, when the object 15 is, for example, a human body and it is difficult to arrange the plurality of transducers 17 on all closed surfaces that surround the object 15, it is desirable to arrange the plurality of transducers 17 on the surface (hemispherical surface) of the hemispherical support member 22 having an opening as in the present embodiment.

It is desirable that the plurality of transducers 17 on the support member 22 be arranged such that sampling can be performed at equal intervals in a k-space. For example, it is desirable that the plurality of transducers 17 be arranged in a spiral pattern as described in U.S. Pat. No. 5,713,356.

In general, a normal direction to a receiving surface (front surface) of a transducer is a direction of highest receiving sensitivity. By causing axes (hereunder referred to as “directional axes”) along the directions of highest receiving sensitivity of the plurality of transducers 17 to gather towards the center of curvature of the hemisphere, a region that can be formed into a visible region is formed with high precision near the center of curvature. Particularly, in the present embodiment, the plurality of transducers 17 are arranged such that the directional axes of the respective transducers intersect the center of curvature of the hemisphere. This can increase the resolution of a region where the directional axes gather. In the description, such a region of high resolution is referred to as a high-resolution region 23. In the present embodiment, the high-resolution region 23 refers to a region that extends from the point of highest resolution to the point at which the resolution is half the highest resolution. Note that as long as the directional axes gather to a specific region and the desired high-resolution region 23 can be formed, the directional axes of the respective transducers need not intersect with each other.

FIG. 1 illustrates an exemplary arrangement of the transducers, and the way of arrangement of the transducers is not limited thereto. The transducers may be arranged in any way as long as the directional axes can be gathered to a specific region and a desired high-resolution region can be formed. That is, the plurality of transducers 17 may be arranged along a curved shape so as to form a desired high-resolution region. Further, in the description, the term “curved surface” also refers to a spherical surface or a spherical surface having an opening, such as a hemispherical surface. In addition, the term “curved surface” also refers to an uneven surface that is uneven to the extent that allows it to be considered as a spherical surface and a surface of an ellipsoid (which is a three-dimensional analog of an ellipse and has a two-dimensional curved surface) that is elliptical to the extent that allows it to be considered as a spherical surface.

When the plurality of transducers 17 are arranged along the support member 22 having a shape formed by arbitrarily sectioning a sphere, the directional axes maximally gather at the center of curvature of the support member. The hemispherical support member 22 that is described in the embodiment is also an example of a support member having a shape formed by arbitrarily sectioning a sphere. In the description, a shape that is formed by arbitrarily sectioning a sphere refers to a shape based on a sphere. Accordingly, the plurality of transducers that are supported by the support member having such a shape based on a sphere are supported on a sphere.

For example, other curved or piecewise linear surfaces can also be used as the support member 22.

It is desirable that the support member 22 have a space that can be filled with the acoustic matching material 18.

### Acoustic Matching Material 18

The acoustic matching material 18 is an impedance matching material that can fill a space between the object 15 and the plurality of transducers 17 and that acoustically couples the object 15 and the plurality of transducers 17. As the acoustic matching material 18, it is desirable to use a material whose acoustic impedance is close to those of the object 15 and the transducers 17 and that transmits pulsed light therethough. In addition, it is desirable that the acoustic matching material 18 be a liquid or a gas that does not prevent the movement of the support member 22. More specifically, the acoustic matching material 18 may be, for example, water, castor oil, or gel.

### Computer 19

The computer 19 is capable of performing predetermined processing on electric signals output from the plurality of transducers 17. The computer 19 is capable of controlling the operation of each component of the photoacoustic apparatus. The computer 19 is capable of setting a desired measurement position. That is, the computer 19 is capable of providing a desired measurement position by controlling the timing in which the light source 11 emits light and driving of the scanner 21 that moves the support member 22.

A computing unit in the computer 19 typically includes an element, such as a central processing unit (CPU), a graphics processing unit (GPU), or an analog-to-digital (A/D) converter; or a circuit, such as a field programmable gate array (FPGA) or an application specific integrated circuit (ASIC). The computing unit may be formed not only by a single element or circuit, or but also by a plurality of elements or circuits. Also, each processing operation performed by the computer 19 may be performed by any of the elements or circuits.

The storage unit in the computer 19 typically includes a storage medium, such as a read-only memory (ROM), a random-access memory (RAM), or a hard disk. The storage unit may be formed not only by a single storage medium, but also by a plurality of storage media.

It is desirable that the computer 19 be configured to perform pipeline processing of a plurality of signals at the same time. This can reduce the time necessary to acquire object information.

Each processing operation performed by the computer 19 can be stored in the storage unit as a program to be executed by the computing unit. Note that the storage unit where the program is stored is a non-transitory recording medium.

### Display Device 20

The display device 20 is a device that displays image data output from the computer 19. Although a liquid crystal display or the like is typically used as the display device 20, a plasma display, an organic electro-luminescent (EL) display, or a field emission display (FED) may also be used. The display device 20 may be provided separately from the photoacoustic apparatus.

### Scanner 21

The scanner 21, serving as a moving unit, moves the support member 22 relative to the object 15. Since the plurality of transducers 17 arranged on the support member 22 can be moved relative to the object 15 by the scanner 21, the photoacoustic waves 16 can be received at a plurality of measurement positions.

It is desirable that the scanner 21 cause the support member 22 to undergo circular movement. The term “circular movement” refers to a curvilinear movement similar to a circular movement and an elliptical movement. It is desirable that the scanner 21 move the support member 22 such that coordinates in a radial direction with respect to the center of a movement region either increase or decrease.

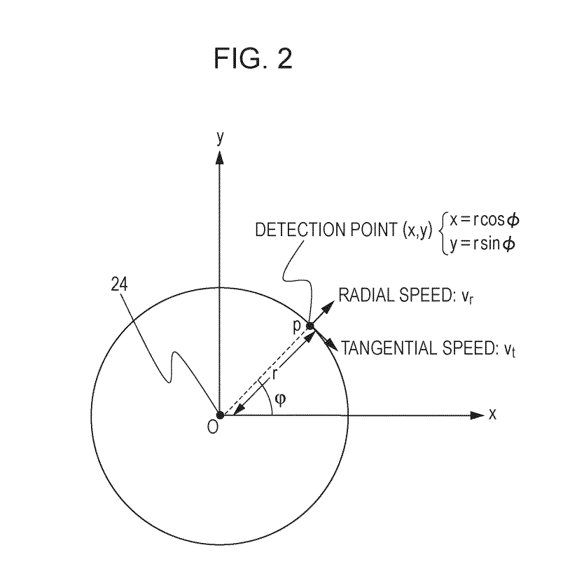

FIG. 2 schematically illustrates an exemplary circular movement. Referring to FIG. 2, a point o is a movement plane center 24, a circle represents a movement path of a position of the support member 22, and a point p is a point on the movement path of the position of the support member 22. This movement gives a speed in a radial direction (radial speed) vᵣ and a speed in a tangential direction (tangential speed) vₜ to the position of the support member 22 at the point p. Position coordinates (x, y) of the point p in a polar coordinate system can be expressed by Equation (1) below:

$$\begin{matrix}[ Math . 1 ] & \\ \{ \begin{matrix}x = r cos φ \\ y = r sin φ\end{matrix} & Equation ( 1 )\end{matrix}$$

where r is a coordinate in the radial direction (movement radius), and φ is an angle formed between the x axis and a line extending from the origin to the point p. In the present embodiment, the scanner 21 moves the support member 22 such that coordinates (r) on the movement path of the position of the support member 22 in the radial direction either increase or decrease.

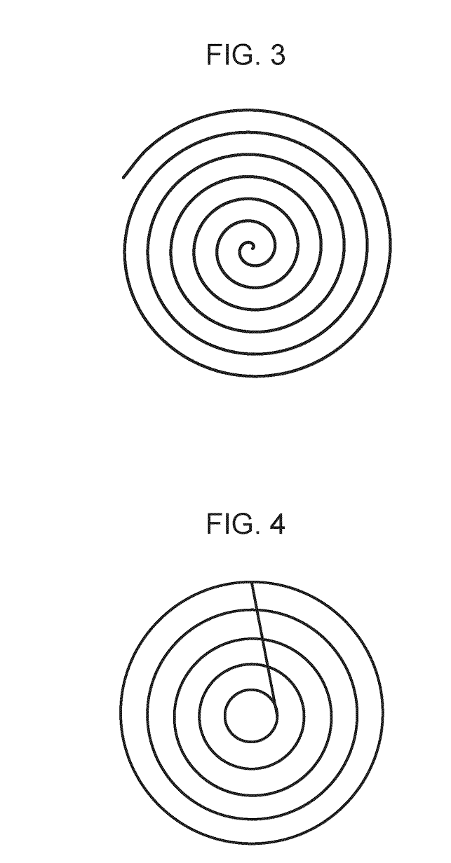

Specific examples of the movement path include a spiral movement path such as that shown in FIG. 3 in which the radius changes with time and a movement path such as that shown in FIG. 4 including a plurality of concentric circles with different radii.

The acoustic matching material 18 with which a container of the support member 22 is filled is subjected to inertial force due to the movement of the support member 22. When the support member 22 undergoes linear movement, if the direction is repeatedly changed, the acoustic matching material 18 may become foamy as a result of a change in a liquid level due to the inertial force. Therefore, a location between the object 15 and the plurality of transducers 17 may not be filled up with the acoustic matching material. In contrast, when the support member 22 undergoes circular movement, the acoustic matching material 18 is subjected to a force in an outer peripheral direction of the circular movement at all times. Therefore, compared to a movement pattern formed by the linear movement in which the direction is repeatedly changed, the circular movement makes it possible to gradually change the liquid level. Therefore, acoustic matching between the object 15 and the plurality of transducers 17 is facilitated.

When the support member 22 is caused to undergo circular movement, the number of sudden accelerations and decelerations is small. Therefore, it is possible to restrict the movement of the acoustic matching material, and to maintain good acoustic matching between the object 15 and the plurality of transducers 17.

It is desirable that the scanner 21 move the support member 22 such that the speed in a direction tangent to the movement path is constant. When the light source 11 is a pulsed light source that emits light at a constant period, the timing of measuring the photoacoustic waves 16 is determined by the repetition frequency of the pulsed light 12 emitted from the light source 11. For example, if the light source 11 has a repetition frequency of 10 Hz, the photoacoustic waves 16 can be generated once every 0.1 seconds. Therefore, if the tangential speed is constant and the photoacoustic waves 16 are measured every 0.1 seconds, the measurement positions are spatially uniformly distributed.

It is desirable that the scanner 21 move the support member 22 from the outer side of the movement plane, in consideration of the acceleration toward the origin. That is, if the acceleration in the initial stage of the movement is large, the magnitude of vibration of the entire apparatus may increase and the vibration may affect the measurement. Therefore, when the support member 22 starts moving from an outer periphery where the acceleration toward the origin is small, and then moves towards an inner periphery, the vibration of the apparatus can be reduced.

It is desirable that the scanner 21 continuously move the support member 22, instead of moving it in a step-and-repeat manner where the support member 22 is moved and stopped repeatedly. This can reduce the overall time required for the movement and reduce the burden on a person being examined. Since the change in acceleration of movement is small, the influence of vibration of the apparatus or the influence of vibration of the acoustic matching material 18 can be reduced.

To move the position of irradiation performed using the pulsed light 12 generated from the light source 11, it is desirable that the scanner 21 move the optical system 13 together with the support member 22. That is, it is desirable that the scanner 21 move the support member 22 and the optical system 13 in synchronism with each other. Thus, since the relationship between the photoacoustic-wave measurement position and the light irradiation position can be kept constant, uniform object information can be acquired. If the object 15 is a human body, the irradiation area for irradiating the object 15 is limited by an American National Standards Institute (ANSI) standard. Therefore, although it is desirable that the irradiation intensity and the irradiation area be increased to increase the amount of light propagating to the interior of the object 15, the irradiation area is limited, from the viewpoint of, for example, reducing the cost of the light source. Because of the directionality of transducers, the efficiency with which light is used is low even if the light is applied to a region of low reception sensitivity. That is, irradiating the entire object of large size is not efficient. Since the efficiency with which light is used is good if light is applied at all times to a region where the sensitivity of the plurality of transducers 17 is high, it is desirable to move the scanner 21 while maintaining the positional relationship between the plurality of transducers and the optical system 13.

In the embodiment, a light-outgoing portion of the optical system 13 is disposed at the center (polar portion) of the support member 22 to apply the pulsed light 12 towards the center of curvature of the support member 22. This causes light to be applied at all times to a region where the sensitivity of the plurality of transducers 17 is high. Since the support member 22 and the optical system 13 are integrated to each other, it is possible to move a high-resolution region while maintaining the aforementioned relationship between the photoacoustic-wave measurement position and the light irradiation position.

The computer 19 can control the magnitude of movement, such as the maximum value of the coordinates r in the radial direction, the speed of movement (i.e., the radial speed and the tangential speed), and the way of changing the coordinates in the radial direction. It is desirable that the maximum value of the coordinates r in the radial direction be changed in accordance with the size of the object. For example, when the object is small in size, the movement of the support member 22 can be controlled with small coordinates r, whereas when the object 15 is large in size, the movement of the support member 22 can be controlled with large coordinates r. This can reduce excess measurement time.

It is desirable that the photoacoustic apparatus include a size acquiring unit capable of acquiring information regarding the size of the object 15. For example, a charge-coupled device (CCD) sensor capable of acquiring information regarding the shape of the object 15 may be used as the size acquiring unit. The computer 19 may determine the maximum value of the coordinates r in the radial direction in accordance with information regarding the size of the object 15 acquired by the size acquiring unit.

It is desirable that the photoacoustic apparatus include an input unit that allows a user to specify a movement parameter, such as the maximum value of the coordinates r in the radial direction, for the computer 19.

By applying the pulsed light 12 at a plurality of timings after the scanner 21 has moved the support member 12, high-resolution regions exist at different positions on the basis of respective measurement timings. As a result, an area of high resolution regions is expanded. In order to reduce variations in the resolution in a region that is subjected to imaging, it is desirable to move the support member 22 so that the plurality of high-resolution regions overlap.

### Distribution of Measurement Positions

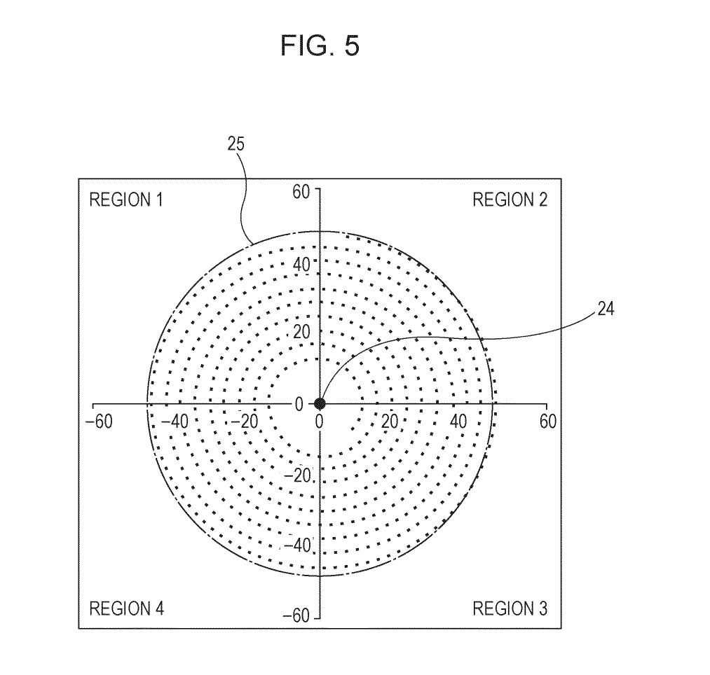

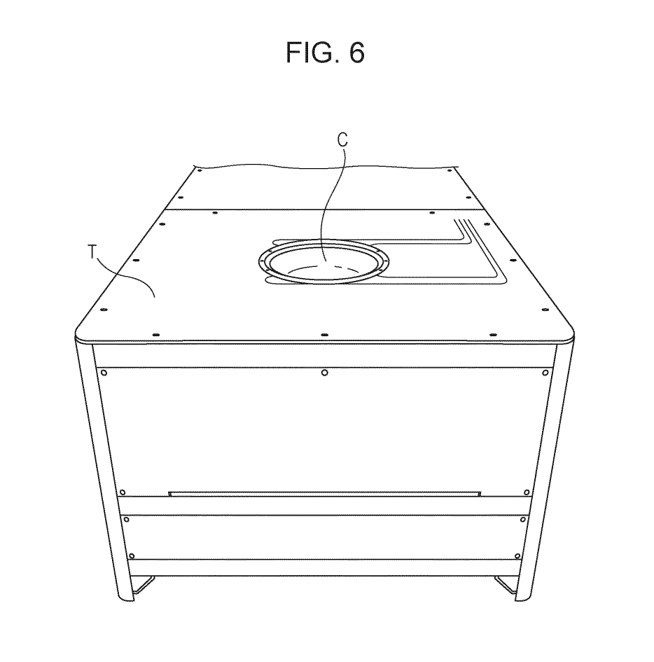

Referring to FIG. 5, a center position (measurement position) of the support member 22 at each light irradiation timing (measurement timing) in the photoacoustic apparatus according to the embodiment is plotted. A point where a vertical line intersects the support member 22 is defined as the center position, that is, the measurement position of the support member 22, with the vertical line extending downward from the center of a high-resolution region 23 to a movement plane. When the support member 22 is hemispherical as in the embodiment, the polar portion of the hemisphere is defined as the center position of the support member 22. Note that when the position of each transducer at the corresponding light irradiation timing is plotted, its distribution is identical to that illustrated in FIG. 5. That is, the distribution of center positions of the support member 22 can explain the positions that can be taken by each of the plurality of transducers 17 at respective measurement timings.

In the photoacoustic apparatus according to the embodiment, the computer 19 sets a movement path of the support member 22 and a light irradiation timing (measurement timing) such that the measurement positions are distributed as shown in FIG. 5. That is, the computer 19 sets the movement path of the support member 22 and the light irradiation timing so that the measurement positions are distributed in such a manner that the density of the measurement positions in the movement region becomes constant as shown in FIG. 5. In accordance with the set distribution of measurement positions, the scanner 21 moves the support member 22, and the light source 11 generates the pulsed light 12 when the support member 22 is positioned at a set measurement position. The photoacoustic apparatus in the present embodiment increases the density of projections while simultaneously increasing the field of view (FOV). By adjusting the number of projection angles in proportion to the projected area encompassed, we were able to maintain contrast sensitivity and spatial resolution for arbitrarily large fields of view.

The photoacoustic apparatus according to the present embodiment is capable of efficiently and precisely acquiring object information for each reconstruction position in a region subjected to imaging.

For example, with the number of elements and the number of measurements being the same, as compared to the case of moving a plurality of transducers whose directional axes are parallel to each other, the photoacoustic apparatus according to the present embodiment is capable of acquiring more wave-number information regarding a photoacoustic wave generated at each reconstruction position.

However, due to an error in light irradiation timing, the arrangement of the plurality of transducers 17, the movement of the support member 22, or the like, it is difficult to receive photoacoustic waves having exactly the same wave-number vector generated from each reconstruction position. Therefore, as long as photoacoustic waves propagating substantially in the same direction from each reconstruction position can be received, it is possible to determine that data having the same relationship in k-space for each reconstruction position can be obtained. For example, when a direction is expressed in terms of its solid angle, acoustic waves that propagate in directions within ±4/50πC steradians of the propagation direction of a photoacoustic wave having a given wave-number vector can be defined as photoacoustic waves having the same wave-number vector.

In the present embodiment, the plurality of transducers 17 are three-dimensionally arranged on the support member 22. Therefore, even when the support member 22 is moved within a limited range in two dimensions, acoustic waves propagating in all directions from each reconstruction position can be received. That is, by moving the support member 22 as shown in FIG. 5, the distribution of the positions that can be taken by the plurality of transducers 17 at all measurement positions becomes a distribution of positions where acoustic waves (having the same wave-number vector) that propagate in substantially the same direction from the respective reconstruction positions can be detected. Therefore, object information for each reconstruction position can be acquired by using data of acoustic waves obtained by receiving acoustic waves having the same relationship in k-space from data acquired at different measurement timings. This makes it possible to acquire object information for each voxel using received signal data of photoacoustic waves that have propagated in all directions from each voxel. Therefore, the reproducibility of acquired object information is high. As regards the acquired object information, since the uniformity of pieces of data used in acquiring the object information for each reconstruction position of a region that is subjected to imaging is high, variations in resolution in the region that is subjected to imaging are small.

The distribution of measurement positions shown in FIG. 5 is hereunder described in detail.

In the present embodiment, the computer 19 sets measurement positions so as to be distributed spatially uniformly over 360° with respect to the center 24 of the movement plane. In the present embodiment, the movement plane center 24 refers to the center of gravity of a region 25 defined by connecting the measurement positions at an outermost periphery illustrated in FIG. 5.

The positions that can be taken by a given element in the plurality of transducers 17 at respective measurement timings are distributed in a plane parallel to the movement plane of the support member 22. The positions that can be taken by the given transducer 17 are spatially uniformly distributed over 360° with respect to the center of distribution of these positions. Here, the center of distribution of the positions that can be taken by the given element refers to the center of gravity of a region defined by connecting the outermost peripheral positions in the distribution of the positions that can be taken by the given element.

The positions that can be taken by each element of the plurality of transducers 17 are also distributed in a plane that is parallel to and different from a plane in which a cluster of positions that can be taken by the given element selected in the previous description are distributed.

That is, spatially uniformly distributing the plurality of measurement positions over 360° with respect to the center 24 of the movement plane is equivalent to spatially uniformly arranging each element over 360° in a plane that is parallel to the movement plane.

The directions from the respective reconstruction positions towards the transducers differ from each other. That is, the transducers receive with good sensitivity different wave-number vectors of acoustic waves generated at the respective reconstruction positions.

That is, the distributions of measurement positions within four regions (regions 1 to 4) are uniform. The four regions are defined by two orthogonal straight lines passing through the movement plane center 24 shown in FIG. 5. In any of the four regions, the distances between adjacent measurement positions are equal to each other.

When the distributions of measurement positions of in the four regions are compared, the numbers of measurement positions are the same. In addition, the average distances between adjacent measurement positions in all the regions are the same.

Due to error in movement of the support member 22, error in light irradiation timing, or the like, it is difficult to make the distances between adjacent measurement positions exactly the same. The cases where approximately the same effects can be achieved are also within the scope of the present invention. That is, the case where the distances between adjacent measurement positions are the same also includes the case where the distances between adjacent measurement positions are within ±20% of the average distance between adjacent measurement positions.

Errors may also occur in the numbers of measurement positions between regions due to, for example, acceleration or deceleration of the speed of movement of the support member 22 near start and end points of the movement of the support member 22. Therefore, the phrase “the numbers of measurement positions are the same” means that the average difference in the numbers of measurement positions between the regions is within ±20%. In addition, the phrase “the distances between adjacent measurement positions are the same” means that the average difference in distances of adjacent measurement positions between the regions is within ±20%. That is, if the average difference in the numbers of measurement positions between the regions and the average difference in the distances of adjacent measurement positions between the regions are both within ±20%, the distribution of center positions of the support member 22 in these regions can be considered uniform.

It is desirable that the relationship described above be established even in the four regions defined by any two orthogonal straight lines passing through the center of the movement plane. The present embodiment has described the case where measurement positions are distributed in a plane. The present invention also includes the case where the movement path extends in a three-dimensional movement region, and the relationship described above is established in the four regions defined by two orthogonal planes passing through the center of the movement region.

It is desirable that a plurality of measurement positions be distributed at different positions in any of the regions. This means that high-resolution regions 23 exist at a plurality of different positions in the regions. Therefore, variations in resolution in the regions 1 to 4 are reduced. Thus, it is possible to suppress an imbalance in resolution depending upon position even in an entire region that is subjected to imaging.

It is desirable that a plurality of measurement positions be set such that each measurement position be spatially equidistance from at least three measurement positions adjacent thereto so as to be disposed at equal intervals. By this, in a region that is subjected to imaging, in an area where there are high-resolution regions at measurement positions that are distributed at equal intervals, intervals between adjacent high-resolution regions are equal, so that variations in resolution are reduced.

It is desirable that the light source 11 emit the pulsed light 12 at a constant repetition frequency, and the support member 22 be moved at a constant speed. In this case, pairs of adjacent measurement positions in the direction of movement along the movement path are disposed at equal intervals. Additionally, it is desirable that the movement path and the measurement timing be set such that the interval between two adjacent measurement positions along the movement path and the interval between adjacent measurement positions in a direction that is different from the movement direction along the movement path be equal.

However, due to an error in movement of the support member 22, an error in light irradiation timing, or the like, it is difficult to make the distances between measurement positions exactly the same. The cases where approximately the same effects can be achieved are also within the scope of the present invention. That is, the term “equal interval” refers to the case where distances to at least three center positions adjacent to a given measurement region are all within ±10% of the average distance to the at least three measurement positions.

In the present embodiment, the case in which measurement positions are distributed in a plane is described. The present invention also includes the case where the movement path extends in a three-dimensional movement region, and a measurement position that is equidistant from at least three measurement positions adjacent thereto is distributed in the three-dimensional movement region.

It is not necessary for the same wave-number component to be efficiently obtained in all regions that are subjected to imaging. All that is required is for the same wave-number component to be efficiently obtained at each reconstruction position of some of the regions that are subjected to imaging. That is, all that is required is that there exist a region in which the density of the distribution of the measurement positions in the movement region is constant. For example, it is possible for a user to set a region of interest using the input unit and to set a measurement position that allows the same wave-number component to be efficiently obtained for each reconstruction position within the region of interest. That is, all that is required is to set the measurement positions so that the density of distribution of measurement positions for a movement region corresponding to a region of interest within the movement region is constant.

### EXAMPLE

A photoacoustic apparatus to which the present embodiment is applied, the photoacoustic apparatus being an apparatus that realizes photoacoustic imaging, will now be described. A human breast will be measured in this example.

The PAI scanner, pictured in FIG. 6, consists of an exam table (T) upon which a patient lies prone, placing one breast in a spherically shaped cup (C), thermoformed from a 0.020″ thick sheet of Polyethylene terephthalate (PETG). A small amount of water is placed in the breast cup along with the breast prior to imaging to provide acoustic coupling between the breast and the breast cup.

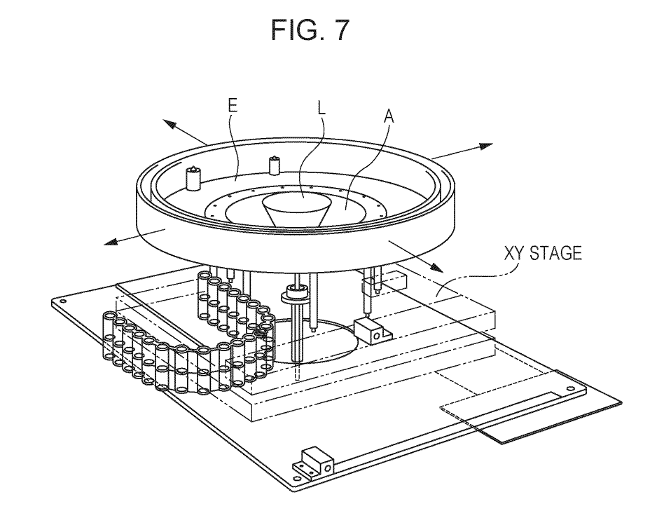

FIG. 7 shows the hemispherical detector array (A), which lies beneath the cup, affixed to a two-axis translational stage (XY), whose position is controlled by a pair of computer-controlled, synchronous motors. The detector array is comprised of a hemispherical shell (radius=127 mm), machined from ABS plastic, in which are embedded 512 discrete transducer elements. Each transducer has a flat active area with a 3-mm diameter. The center frequency of the 1-3 piezo-composite transducers is 2 MHz with a 70% bandwidth.

The detector array and a plastic extension (E) to the array were filled with degassed, RO water to provide acoustic coupling between the breast cup and the 512 transducers. A 7-mm-diameter, pulsed Alexandrite laser beam (75 ns @ 300 mJ/pulse) was fed through an articulating arm that directed the laser beam (L) upward along the vertical axis of the transducer array as the array was scanned. A −12 mm diverging lens, placed at the base of the array, spread the light in a conical fashion to a diameter of ˜60 mm at the surface of the breast cup. The peak light fluence was measured as ˜10 mJ/cm2 at the center of the beam, which is less than half the maximum permissible exposure (MPE) recommended by the ANSI.

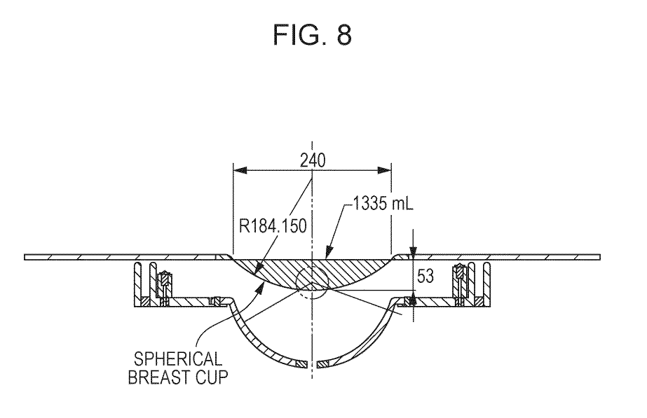

A cutaway of the PAI scanner, which shows the geometric relationships among the detector array, array extension, imaging table, and breast cup, is illustrated in FIG. 8. The array extension allows the detector array to be scanned laterally across the breast surface and still maintain water coupling to the breast. The maximum imaging volume (1335 mL) is defined by the radius of curvature of the breast cup (184 mm), the width of the aperture through which the breast is placed (240 cm) and the maximum penetration depth for which hemoglobin can be visualized. This maximum imaging volume is denoted by the hatching in FIG. 8.

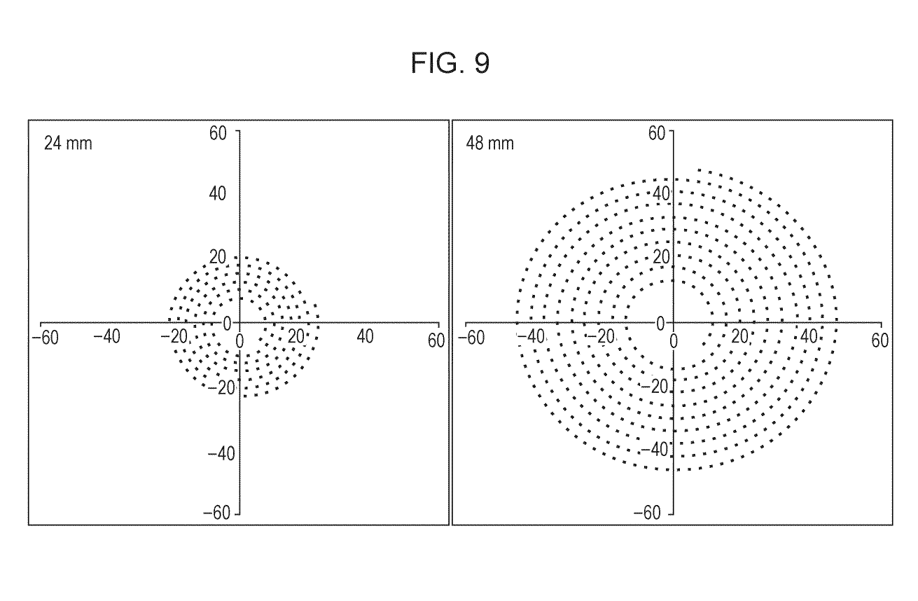

For this PAI scanner we scanned the detector array continuously in a spiral pattern within a plane that lay normal to the rotational axis of the array as the laser beam was pulsed at 10 Hz. The spiral patterns we chose were such that the PA data are acquired at locations within a plane that are spaced equidistantly, the spacing being the same no matter the size of the spiral. Thus larger spirals required more pulses and longer image-acquisition times than smaller spirals. Examples of two of the spiral patterns used for this work are illustrated in FIG. 9. The smallest spiral had a maximum radius of 24 mm and consisted of 120 discrete locations; the larger spiral had a radius of 48 mm and consisted of 480 discrete locations.

Data from each of the 512 transducers were digitized to 12 bits at 20 MHz for a total of 2048 samples following each laser pulse. Total data acquisition time was anywhere from 12 seconds for the smallest spiral to 3.2 minutes for the largest spiral (96 mm). The lateral field of view (FOV) varied from 100 mm to 240 mm diameter, depending on the exact spiral pattern chosen.

Three-dimensional PAI images were reconstructed using a filtered-backprojection algorithm that has been described previously. We first measured the PA response to a “point” absorber fabricated from a small spot of ink placed on the tip of a clear, thin polyethylene thread, which was then used to calculate a ramp filter function and simultaneously deconvolve the impulse response of our transducers. After filtering all our data, we backprojected our temporal data over spherical surfaces, the radii of which were determined from the measured speed of the sound of the water coupling and a second, assumed speed of sound within the breast (or phantom). We assumed that everything above the breast cup was homogeneous phantom or breast tissue, and everything beneath was water. The speed of sound of water was calibrated in our laboratory as a function of temperature, which was recorded during data acquisition. We chose the sound speed within the breast (phantom) interactively by visually assessing the “sharpness” of vessels within the breast as the assumed sound speed above the breast cup was varied.

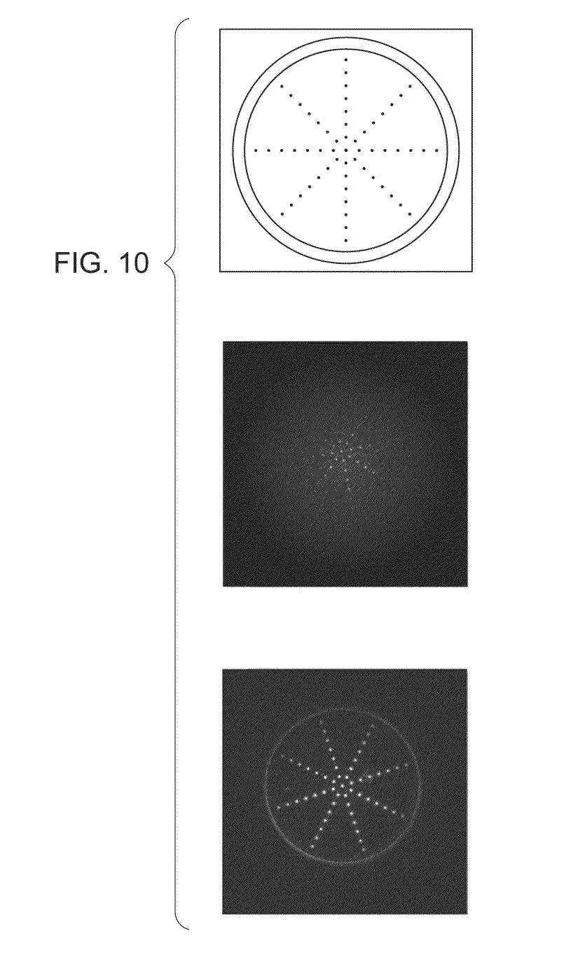

By using a contrast phantom shown in the upper part of FIG. 10, an experiment was performed to determine the FOV expansion effect achieved by moving the detector array. It consisted of an array of 1-mm, dots printed out on a disk of clear plastic with an HP laser printer and affixed to a 1-cm thick disk of polyvinylchloride-plastisol (PVCp) to provide rigidity. The absorbance of the ink at 756 nm was measured with a Genesys 10vis spectrophotometer as 0.129, which is approximately the same absorbance one expects for a 1-mm thick sample of blood containing 150 g/L of oxyhemoglobin. We therefore thought it a good surrogate for blood absorption by a 1-mm-thick blood vessel. We filled the breast cup with an 8% solution of stock Liposyn-20% to simulate the attenuation of breast tissue. A photoacoustic image acquired without moving the detector array is shown in the middle part of FIG. 10. As can be seen from the photoacoustic image in the middle part of FIG. 10, the phantom was not entirely imaged and the imaged region (FOV) was small. Next, the detector array was moved in a spiral pattern shown in the left part of FIG. 9, and a photoacoustic image shown in the lower part of FIG. 10 was acquired. As can be seen from the photoacoustic image in the lower part of FIG. 10, the image of the entire phantom was acquired and the FOV was expanded by moving the detector array.

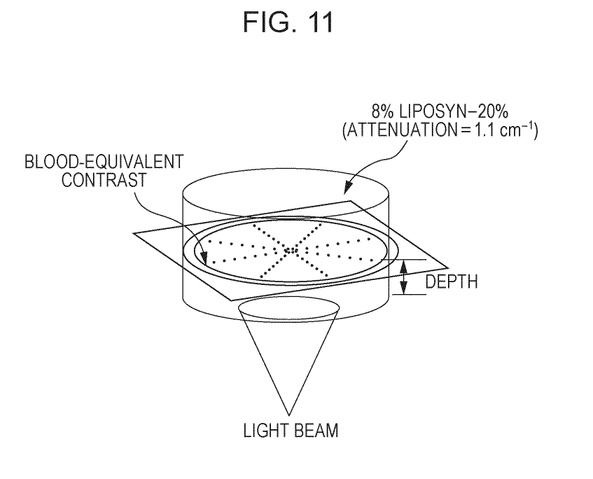

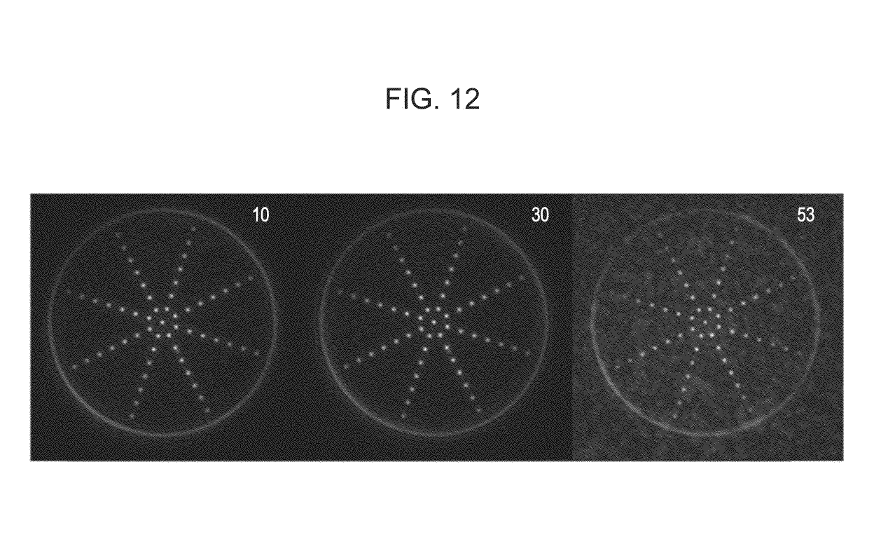

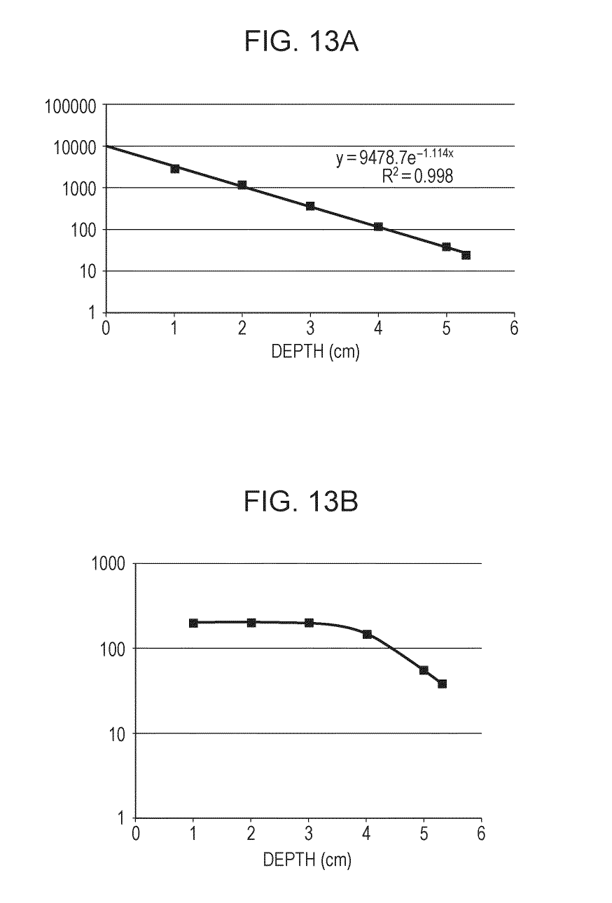

We quantified the contrast-to-noise performance of the PAI system by imaging the phantom shown in the upper part of FIG. 10. We also filled the breast cup with an 8% solution of stock Liposyn-20% to simulate the attenuation of breast tissue. The contrast phantom was placed at varying depths within the Liposyn solution as shown in FIG. 11. PAI images of the phantom were made using a 24-mm spiral scan (12 seconds). PAI images at three depths of Liposyn solution are shown in FIG. 12. The contrast and noise were measured from such images as a function of depth of Liposyn solution between the breast cup and the location of the contrast phantom. The contrast and contrast-to-noise ratios were then plotted as a function of depth of Liposyn solution and analyzed. The photoacoustic contrast for the center “dot” of our contrast target (FIG. 11) was measured and plotted as a function of the depth of the 8% Liposyn solution. The result is plotted in FIG. 13A and displays a nearly perfect exponential decay with depth. The noise was calculated as the standard deviation within a small region of the background in the contrast target. This “noise” estimate was recorded and used to calculate the contrast-to-noise ratio (CNR), which is plotted in FIG. 13B.

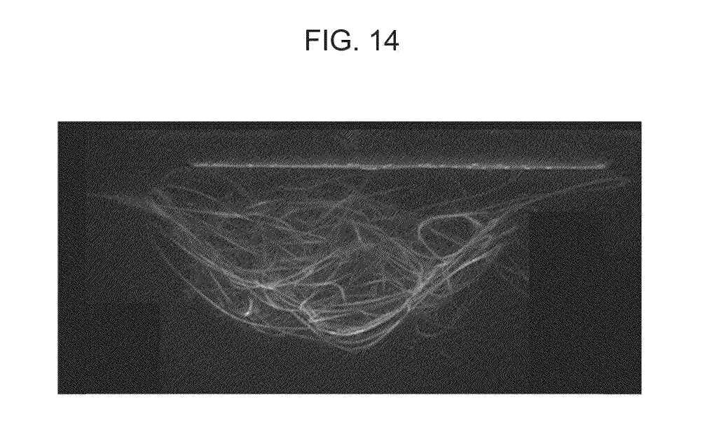

We note that the CNR remained constant through the first 3.0 cm of turbid media before dropping, as the depth increased. The reason for this behavior is due to the dominance of “streak noise”, which is much greater than the electronic noise “floor” of the input electronics of our data acquisition system at shallow depths. As the PAI contrast falls to lower levels at greater depths, this electronic noise begins to degrade our CNR as can be seen in FIG. 13. We measured the spatial resolution of the PAI scanner by imaging a long filament of graphite fiber embedded in an agar mold, shaped to fit snugly into our breast cup. A MIP calculated from a 3D PAI image (48-mm spiral scan, 48 seconds) are shown in FIG. 14. The full-width half-maximum (FWHM) of a profile across one of the carbon fibers was used to estimate the spatial resolution of the PAI system. The spatial resolution was estimated from a plot across one filament of the graphite fiber phantom (FIG. 14). The full-width, half-maximum of the plot was 0.42 mm.

The resolution was kept almost constant throughout substantially the entire region within the FOV.

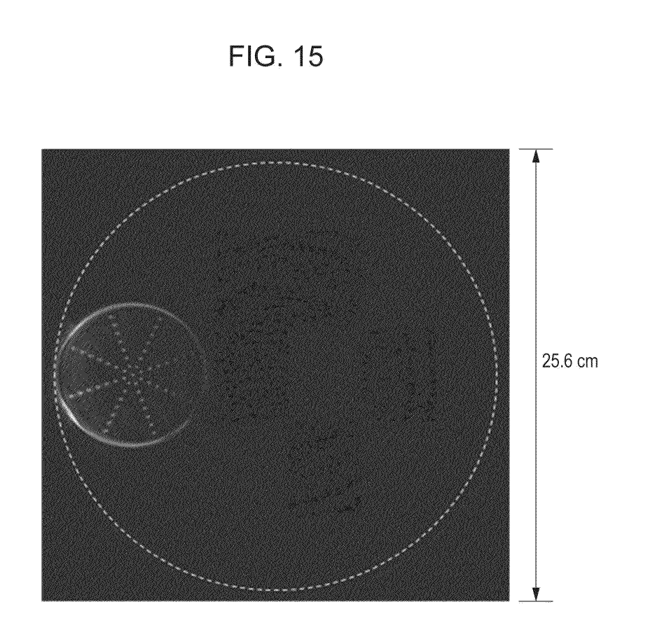

In order to demonstrate that we can maintain good contrast sensitivity at the periphery of the field of view, we translated our contrast phantom laterally 80 mm and scanned with our largest spiral protocol (96 mm, 3.2 minutes). The lateral field of view was measured as 24 cm as indicated from the visibility of the contrast phantom placed at the edge of our field of view (FIG. 15). The lateral field of view was measured as 24 cm as indicated from the visibility of the contrast phantom placed at the edge of our field of view (FIG. 15).

We chose to scan the hemispherical detector array continuously in a spiral pattern during data acquisition, rather than rotating the array about its vertical axis as had been done in our previous prototype PAT scanner. This new spiral scan protocol increased the density of projections while simultaneously increasing the FOV. By adjusting the number of projection angles in proportion to the projected area encompassed by the spiral scan, we were able to maintain contrast sensitivity and spatial resolution for arbitrarily large fields of view as needed in breast imaging.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. Patent Application No. 61/873,542, filed Sep. 4, 2013, which is hereby incorporated by reference herein in its entirety.

1. A photoacoustic apparatus comprising:
a light source;
a plurality of transducers configured to detect acoustic waves and output electric signals, the acoustic waves being generated when an object is irradiated with light generated from the light source;
a support member configured to support the plurality of transducers such that directional axes of the plurality of transducers gather;
a moving unit configured to move the support member relative to the object within a movement region;
a storage unit configured to store the electric signals output from the plurality of transducers at a plurality of timings; and
a computing unit configured to acquire object information for each reconstruction position on the basis of the electric signals stored in the storage unit,
wherein the light source generates the light at the plurality of timings, and
wherein the moving unit moves the support member such that there exists a region in which a density of a distribution of positions of the support member at the plurality of timings is constant.

2. The photoacoustic apparatus according to claim 1, wherein the moving unit moves the support member such that distances between adjacent positions of the support member among the positions of the support member at the plurality of timings are equal to each other.

3. The photoacoustic apparatus according to claim 2, wherein the moving unit moves the support member such that the distances between the adjacent positions of the support member are within ±20% of an average distance between the adjacent positions of the support member in the movement region.

4. The photoacoustic apparatus according to claim 1, wherein the moving unit moves the support member such that distances from each position of the support member at the plurality of timings to at least three positions of the support member adjacent thereto are equal to each other.

5. The photoacoustic apparatus according to claim 4, wherein the moving unit moves the support member such that the distances from each position of the support member at the plurality of timings to the at least three positions of the support member adjacent thereto are within ±10% of an average of the distances.

6. The photoacoustic apparatus according to claim 1, wherein the moving unit moves the support member such that a distribution of positions of the support member at a light irradiation timing in each of four regions defined by two orthogonal planes passing through a center of the movement region is uniform.

7. The photoacoustic apparatus according to claim 6, wherein the moving unit moves the support member such that an average difference in the number of positions of the support member at the light irradiation timing in each of the four regions is within ±20%, and an average difference in distance between adjacent positions of the support member is within ±20%.

8. The photoacoustic apparatus according to claim 1, wherein the moving unit moves the support member such that in each of four regions defined by two orthogonal planes passing through a center of the movement region, the number of positions of the support member at a light irradiation timing is plural.

9. The photoacoustic apparatus according to claim 1, wherein the computing unit acquires the object information for each reconstruction position using, among the electric signals stored in the storage unit, electric signals output from the plurality of transducers at a part of the plurality of timings.

10. The photoacoustic apparatus according to claim 1, wherein the computing unit acquires the object information for each reconstruction position using, among the electric signals stored in the storage unit, electric signals corresponding to acoustic waves having a same wave-number vector.

11. The photoacoustic apparatus according to claim 1, wherein the moving unit continuously moves the support member.

12. The photoacoustic apparatus according to claim 11, wherein the light that is generated by the light source has a constant period, and
wherein the moving unit moves the support member at a constant speed in a tangential direction of a movement path of the support member.

13. The photoacoustic apparatus according to claim 1, wherein the moving unit continuously moves the support member in a circular movement path.

14. The photoacoustic apparatus according to claim 1, wherein the moving unit moves the support member such that coordinates in a radial direction with respect to a center of the movement region either increase or decrease.

15. The photoacoustic apparatus according to claim 1, wherein the moving unit moves the support member in a spiral-pattern movement path in which a radius increases or decreases with time.

16. The photoacoustic apparatus according to claim 15, wherein the moving unit moves the support member from an outer periphery towards an inner periphery of the spiral-pattern movement path.

17. The photoacoustic apparatus according to claim 1, wherein the moving unit moves the support member in a plurality of concentric movement paths having different radii.

18. The photoacoustic apparatus according to claim 1, wherein the support member is hemispherical in shape.

19. The photoacoustic apparatus according to claim 1, further comprising an optical system configured to guide the light generated from the light source to a position where the directional axes gather,
wherein the moving unit moves the support member and the optical system in synchronism with each other.

20. The photoacoustic apparatus according to claim 1, wherein the support member supports the plurality of transducers such that the plurality of transducers are arranged in a three dimensional space.

21. The photoacoustic apparatus according to claim 1, wherein the moving unit moves the support member so that a density of distribution of positions of the support member at a light irradiation timing corresponding to a set region of interest is constant.

22. A photoacoustic apparatus comprising:
a light source;
a plurality of transducers configured to detect acoustic waves and output electric signals, the acoustic waves being generated when an object is irradiated with light generated from the light source;
a support member configured to support the plurality of transducers such that directional axes of the plurality of transducers gather;
a moving unit configured to move the support member relative to the object within a movement region;
a storage unit configured to store the electric signals output from the plurality of transducers at a plurality of timings; and
a computing unit configured to acquire object information for each reconstruction position on the basis of the electric signals stored in the storage unit,
wherein the support member has a space disposed between the support member and the object and capable of being filled with an acoustic matching material, and
wherein the moving unit continuously moves the support member in a circular movement path.

23. The acoustic apparatus according to claim 22, wherein the moving unit moves the support member in a spiral-pattern movement path in which a radius increases or decreases with time.

24. The acoustic apparatus according to claim 23, wherein the moving unit moves the support member from an outer periphery towards an inner periphery of the spiral-pattern movement path.

25. The acoustic apparatus according to claim 22, wherein the light that is generated by the light source has a constant period, and
wherein the moving unit moves the support member at a constant speed in a tangential direction of a movement path of the support member.
